(19) United States
(12) Patent Application Publication
(10) Pub. No.: US 2023/0122776 A1
(43) Pub. Date: Apr. 20, 2023

(54) ANTIVIRAL AGENT

(71) Applicant: Johannes REINMÜLLER, Wiesbaden (DE)

(72) Inventor: Johannes REINMÜLLER, Wiesbaden (DE)

(21) Appl. No.: 17/914,876

(22) PCT Filed: Mar. 25, 2021
(86) PCT No.: PCT/EP2021/057723

§ 371 Date: Sep. 27, 2022

(30) Foreign Application Priority Data
Mar. 27, 2020 (EP) 20166303.6
Apr. 9, 2020 (EP) 20168964.3

(51) Int. Cl. A61K 31/549; A61K 31/728; A61P 31/14; A61K 45/06

(52) U.S. Cl. CPC A61K 31/549; A61K 31/728; A61P 31/14; A61K 45/06

(57) ABSTRACT

The present invention relates to the use of hydroxymethyl group donors, in particular of hyaluronic acid which contains hydroxymethyl groups, for treating and preventing an infection by enveloped viruses such as coronaviruses, paramyxoviruses, e.g. RS viruses, or orthomyxoviruses, such as influenza viruses, and/or for treating or preventing an inflammatory disease of the respiratory tract, for example COPD, ARDS or cystic fibrosis, in particular an inflammatory disease of the respiratory tract associated with a viral infection.

The present invention relates to the use of hydroxymethyl group donors, in particular of hyaluronic acid which contains hydroxymethyl groups, for treating and preventing an infection by enveloped viruses such as coronaviruses, paramyxoviruses, e.g. RS viruses, or orthomyxoviruses, such as influenza viruses, and/or for treating or preventing an inflammatory disease of the respiratory tract, for example COPD, ARDS or cystic fibrosis, in particular also an inflammatory disease of the respiratory tract associated with a viral infection.

A plurality of diseases of the respiratory tract in humans and in higher-order vertebrates is caused by enveloped viruses, such as influenza viruses (family Orthomyxoviridae), coronaviruses (family Coronaviridae), or RS viruses (Respiratory Syncytial Viruses, family Paramyxoviridae). Half of all of what are referred to as colds or diseases of the respiratory tract of a human, are, however, caused by rhinoviruses (family Picornaviridae). In contrast to the above-mentioned viruses, they do not have lipid envelopes and consist of RNA with a surrounding capsid. (They can be detected by PCR testing in the laboratory, and can thus be distinguished from other viral pathogens).

Said viruses are mainly spread by droplet infection (aerosols) and by contact with contaminated surfaces (smear infections). Infection of the afflicted organism requires the pathogen to penetrate into the specific host cell (entry) or to dock onto the surface thereof, and the activity of viral and/or cell-bound enzymes for infiltration. The prevention of the viral invasion into the host cells can thus prevent the outbreak of the disease.

The course of an infection of the respiratory tract is dependent on individual factors, and ranges from asymptomatic to life-threatening states and Exitus letalis.

Hitherto, no specific medicinal-antiviral treatments are known. In practice, treatment takes place symptomatically. In the case of survival, the different viral diseases leave behind highly specific immunities. Therefore, protection against such diseases can currently only be achieved by a vaccination—provided there is experience with the pathogen and a vaccine. The development of a vaccine generally requires several years.

However, the above-mentioned virus families are subject to constant genomic change, so that over the course of successive disease outbreaks new pathogen variants constantly emerge, for which no vaccine is available in advance. It is therefore of particular interest to develop a treatment method which prevents the absorption of the viruses or the multiplication of absorbed viruses in the organism in a less specific or unspecific manner, and thus prevents the disease onset or a serious disease progression.

This can be achieved by modification of proteins of the virus capsid and/or of the viral envelope and/or of the receptor of the target cell, which are essential, in a reciprocal manner, for docking and penetration of the virus into the target cell. Thus, the penetration of the virus into the target cell on the lung surface, and the multiplication thereof is prevented.

The virus capsid of the mentioned families generally consists of proteins and proteoglycans. The cellular receptors are frequently proteins and proteoglycan with enzyme activity (peptidases, metallocarboxypeptidases, proteases) or sialic acids. The blocking of the enzymatically active domains by transfer of hydroxymethyl groups can thus prevent the penetration of the viral pathogen into the cytoplasm of the target cell, and thus the multiplication and spread thereof.

Secondary amines (arginine residues or histidine residues of serine proteases, or catalytic triads), and amino and/or hydroxyl groups of glycosaminoglycans (including sialic acids), or proteoglycans, are possible as acceptors of the hydroxymethyl groups. Amino groups frequently form the active centres of peptidases/proteases (catalytic triads) which, in most known coronaviruses infections, act as cellular receptors or as viral ligands. They therefore play a key role in infection.

Known are the inhibition of aminopeptidases of the cell surface with alpha-ketoamides, and the blockade of the virus cell contact on the ACE2 receptor by an analogue. Hitherto, no approved active agent (antiviral agent) for the treatment of humans has been derived herefrom.

WO2012/168462 describes that glycosaminoglycan derivatives, which were modified with hydroxymethyl groups, have an excellent anti-infective effect with respect to different types of pathogens. This anti-infective effect is due to the presence of hydroxymethyl groups on the glycosaminoglycan.

It was surprisingly found that hydroxymethyl donors have an antiviral and/or viricidal effect against enveloped viruses, in particular coronaviruses, such as the novel coronavirus SARS-CoV2, paramyxoviruses, such as RS viruses, or orthomyxoviruses such as influenza viruses, or non-enveloped viruses, in particular picornaviruses, such as rhinoviruses.

In this case, the inhibition of functional proteins of the cell surface and/or of the virus capsid including the “spikes” is achieved by the transfer of hydroxymethyl groups, which are brought by means of a suitable carrier molecule, to the location of the current or imminent virus infection—in this case the surfaces of the upper and lower respiratory tract. It is currently assumed that the mechanism of action of a hydroxymethyl group donor is based on the transfer of hydroxymethyl groups to functional groups, i.e. acceptors such as serine proteases on the cell surface of the target cell and/or to acceptors of the virus capsid, e.g. arginine clusters of the “spikes” or glycosaminoglycans.

There is thus a broad treatment spectrum for various viral pathogens and the mutants thereof, provided that they use similar infection routes. In this way, the penetration of said pathogens, e.g. coronaviruses, into the target cell is prevented and/or slowed.

Furthermore, an anti-inflammatory effect of hydroxymethyl donors is useful, which is found in the case of administration to the surfaces of the respiratory tract. Said anti-inflammatory effect is based according to the invention on the transfer of hydroxymethyl groups onto the serine proteases of the inflammatory cascade, which is inhibited thereby.

An object of the invention is therefore the use of a hydroxymethyl group donor as an active agent for treating and preventing an infection by viruses, in particular enveloped viruses such as coronaviruses, paramyxoviruses, e.g. RS viruses, or orthomyxoviruses, e.g. influenza viruses, or non-enveloped viruses such as picornaviruses, e.g. rhinoviruses.

A further object of the invention is the use of a hydroxymethyl group donor as an active agent for treating and preventing inflammatory diseases of the respiratory tract, for example COPD, ARDS or cystic fibrosis, in particular also witch viral infections associated inflammatory diseases of the respiratory tract. The viral infections comprise respiratory infections by enveloped viruses, as described above, but also infections by non-enveloped viruses, such as rhinoviruses.

The principle on which the present invention is based is consistent with findings by Hoffmann et al. (Cell 181 (April 2020), 1-10). According thereto, in the case of an infection SARS-CoV-2 viruses penetrate into a target cell (epithelium), whereby the spike protein of the virus is being cleaved by the cell-bound TMPRSS2 protease. This protease is a serine protease. Serine proteases are essentially involved in many physiological processes, e.g. blood coagulation and inflammation, and exhibit identical active centres (serine-histidine-asparagine acid, respectively so-called catalytic triad). Blocking of TMPRSS2 is currently considered the preferred treatment approach.

Thus, in the case of a SARS-CoV-2 infection, the transfer of hydroxymethyl groups can result in an inhibition of the serine protease TMPRSS2 of the target cell and/or a change in the arginine-rich domains of the viral S protein (“spike”), possibly with the consequence of a general blocking of cleavage by other proteases.

S. Buonvino and S. Melino, in “New Consensus pattern in Spike CoV-2: potential implications in coagulation process and cell-cell fusion.” Cell death discovery 6.1 (November 2020): 1-5, describe the occurrence of a consensus motif in the spike protein of coronaviruses. It is characterised by 13 amino acids, such as cysteine and serine, which have through their end groups —SH or —OH a high affinity to hydroxymethyl groups.

The treatment principle pursued here uses a hydroxymethyl donor, e.g. taurolidine and/or modified hyaluronic acid, and thus, in a manner similar to the serine protease inhibitor camostat mesilate, is based on known, approved pharmaceutical preparations. The mentioned substances act as serine protease inhibitors, and thus antivirally in the sense of entry inhibitors.

In a preferred embodiment, the invention relates to the use of a hydroxymethyl group donor for the prevention or treatment of an infection by coronaviruses, e.g. SARS-CoV-2.

It is assumed that the hydroxymethyl donors according to the invention, in particular taurolidine, can also deactivate the spike protein of coronaviruses by binding to the consensus motif, and thus inhibit the entry of the viruses into target cells, e.g. into cells of the pulmonary alveolae.

From J. Reinmüller, “Beeinflussung der physiologischen und pathologischen Gerinnung durch Taurolidin und Implikationen für die Anwendung”, [“Influencing of the physiological and pathological coagulation by taurolidine, and implications for application”], Zentralblatt Chirurgie, 124 (1999) Suppl. 4: 13-18, it is furthermore known that taurolidine inhibits the activity of the blood coagulation factor X. Factor X is a protein which is involved in the blood coagulation cascade, and also contains the consensus motif.

As of recently, it is known that the production of the spike protein in human cells, upon the onset of the Covid-19 disease and upon vaccination with a genetic vaccine (e.g. an mRNA or vector vaccine) can trigger increased blood coagulation, which is characterised by the formation of blood clots in the blood vessels. The consensus motif is also found on the PEAR1 (platelet endothelial aggregation receptor 1), which induces the adhesion of thrombocytes to the vessel wall.

As a side-effect of a coronavirus vaccination with the AstraZeneca vaccine, currently thromboses in the brain are described, which are said to be caused by aggregation of thrombocytes in the brain veins. Since this complication occurs shortly after vaccination, it is plausible that due to the spike protein formation as a result of the AstraZeneca vaccination, the consensus motif occurs in excess in the organism, e.g. in the blood, and directly or indirectly promotes the PEAR1 effect. This can cause an adhesion of thrombocytes to the vein walls, and thus the development of a thrombosis.

Hydroxymethyl donors, in particular taurolidine, can have an anti-coagulation effect, by inhibiting the spike protein itself and/or the body's own proteins, e.g. factor X and/or PEAR1, and thereby prevent and/or at least reduce formation of blood clots.

This results in a new antivirally acting preparation having a hydroxymethyl group donor as the active agent. Preferred active agents are biopolymers that contain hydroxymethyl groups, e.g. glycosaminoglycans, in particular hyaluronic acid, proteoglycans, or carbohydrates, or low-molecular-weight substances such as taurolidine. The composition is preferably present as an aerosol or rinsing solution for use against the spread of the novel coronavirus in the respiratory tract, and for prophylaxis.

The antiviral composition can expediently already be administered at the time of the first symptoms or in the case of a positive test of a patient on account of the throat swab, i.e. before the onset of the disease or before the appearance of serious symptoms, e.g. inhaled as an aerosol, in order to inhibit the spread of the infection into lower portions of the respiratory tract.

In order to prepare the active agent according to the invention, a carrier substance, e.g. a glycosaminoglycan such as hyaluronic acid, can be reacted with a hydroxymethyl donor, e.g. formaldehyde, whereby hydroxymethyl groups are transferred to the glycosaminoglycan, e.g. hyaluronic acid. Suitable examples for hydroxymethyl donors are aqueous and/or alcoholic formalin solutions and/or paraformaldehyde dispersions. Thus, the carrier substance, e.g. the hyaluronic acid, itself becomes the hydroxymethyl donor in vivo. Other compounds, i.e. with respect to the reaction of the hyaluronic acid, are also possible as hydroxymethyl donors, which are capable of transferring hydroxymethyl groups, such as taurolidine, N-methylolcaprolactam, N-methylolpyrrolidon, N-methylolated ureas or thioureas, methylolated dicyandiamide, methylolated melamine, etc. A preferred hydroxymethyl donor is taurolidine, which is itself known as being antiviral and/or viricidal. Taurolidine is possible as a diluted solution in a concentration e.g. of from 0.01 to 1.0% (w/v), in particular from 0.01 to 0.5% (w/v), or from 0.1 to 0.2% (w/v) as an alternative for wetting surfaces of the respiratory tract. The quick degradation of the molecule in biological tissue means that the half-life is shorter than that of a hydroxymethyl group-containing hyaluronic acid. Due to the potential transfer of hydroxymethyl groups to functional structures of the virus and the cell wall, taurolidine acts in a manner according to the invention, and is therefore included in the invention for the treatment of viral diseases of the respiratory tract caused by the mentioned virus families.

In place of hyaluronic acid, other carrier molecules can also be used, which contain amino and/or hydroxyl groups, such as cellulose and hydroxyethyl starch, or other glycosaminoglycans, proteoglycans, long-chain carbohydrates such as glycogen, starch, polyalcohols such as ethylene glycol, polyethylene glycols or glycerin, also chitin and derivatives thereof (e.g. chitosamine), mono-, di- or oligosaccharides, amino alcohols, and suitable peptides of different compositions and chain lengths.

A quantitative demonstration of cleavable hydroxymethyl groups in the carrier molecule can take place by means of known methods, e.g. by the chromotropic acid reaction or by the Schiff test.

In a preferred embodiment of the invention, the hydroxymethyl group donor is a modified glycosaminoglycan, i.e. a polysaccharide that is formed in a linear manner from repeating modified disaccharides, in particular hyaluronic acid. In this case, the individual disaccharide units consist of an uronic acid which is glycosidically bound 1→3 with an amino sugar, such as N-acetyl glucosamine. The disaccharide units of the chains themselves are glycosidically linked 1→4.

Preferred examples for glycosaminoglycans used according to the invention are glycosaminoglycans which are substituted at amino and/or hydroxyl groups with hydroxymethyl groups. In addition to hyaluronic acid, further examples for suitable glycosaminoglycans are heparin, chondroitin sulfate, dermatan sulfate, and keratan sulfate. According to the invention, chitosamine and poly-N-acetyl-glucosamine are furthermore understood as glycosaminoglycans. The glycosaminoglycan is preferably a hyaluronic acid.

Glycosaminoglycans are typically obtained from protein-containing biological tissues. For example, isolation of hyaluronic acid from cockscombs or from streptococci is known. Natural heparins are extracted inter alia from the small intestine mucosa of pigs. Chondroitin sulfate is largely obtained from the cartilage of cows, pig and sharks. Furthermore, glycosaminoglycans can be produced from genetically modified host organisms, e.g. bacteria cells.

In medicine, hyaluronic acid and derivatives thereof are used for viscoelastic supplementation of joints in the case of arthrosis, for backfilling of tissues, in particular of the dermis, known as a “dermal filler,” and for treating inflammatory diseases of the skin and of the mucosae. For example, WO 2005/067944 describes the treatment and prevention of diseases of the skin and the mucosae caused by herpes and papilloma viruses.

It has surprisingly now been found that glycosaminoglycan derivatives, in which one or more amino groups are substituted with hydroxymethyl groups, have an effect against enveloped viruses such as coronaviruses, RS viruses, or influenza viruses.

The disaccharide units, from which glycosaminoglycans are formed, consist, as mentioned above, of an uronic acid and an amino sugar. In the glycosaminoglycan derivatives according to the invention, substituted with hydroxymethyl groups, a hydroxymethyl group is bound to one or more suitable reactive groups, e.g. nitrogen atoms of amino groups and/or hydroxyl groups. The hydroxymethyl group-containing glycosaminoglycans according to the invention thus comprise characteristic substituents such as —N(R)—CH₂OH, where R can be any desired group, in particular H or acetyl, or —O—CH₂OH. These are preferably bound to amino sugars of the disaccharide units of the glycosaminoglycan. In the case of glycosaminoglycans which contain the amino sugar n-acetyl glucosamine, the n-acetyl group is preferably substituted with a hydroxymethyl group. Within the meaning of the invention, —N(acetyl)-CH₂OH groups are characteristic for these hydroxymethyl group-containing glycosaminoglycans. These compounds surprisingly exhibit a particularly good anti-infective effect.

In the glycosaminoglycan derivatives according to the invention, one or more amino groups are substituted with hydroxymethyl groups. The degree of hydroxymethylation is preferably in the range of from 200:1 (0.5%) to 1:1 (100%), preferably 100:1 (1%) to 2:1 (50%), particularly preferably 20:1 (5%) to 10:1 (10%), in each case on a molar basis. As the hydroxymethylation of the glycosaminoglycans increases, the antiviral and/or viricidal effect of glycosaminoglycans increases.

The hydroxymethyl group-containing glycosaminoglycans exhibit excellent tissue tolerance. They remain for a long time at their site of action, whereby the retention time can be controlled by the selection of the molecular weight of the used glycosaminoglycan and its cross-linking level. For example, depending on the molecular weight and cross-linking level thereof, hydroxymethyl hyaluronic acid remains at the site of action from about 30 minutes up to six months.

Hyaluronic acid is inherently biodegradable. It can have same or different chain lengths and/or molecular weights, it can be short-chain (less than 100 repeating units) or long-chain (over 100) with influence on the duration of action. Cross-linking enables a further extension of the duration of action via reduced biodegradation. Hyaluronic acid is furthermore a component of the bronchial secretion and is actively secreted by the surface cells of the lungs.

Hydroxymethyl group-containing glycosaminoglycans within the meaning of the invention are suitable both in non-crosslinked and in crosslinked form, or as mixtures, for treating infectious diseases. Non-crosslinked or crosslinked hyaluronic acid or mixtures thereof are particularly preferably used.

Non-crosslinked glycosaminoglycans are preferably selected from (i) long-chain glycosaminoglycans having an average molecular weight (weight average) of at least 200 kD and (ii) short-chain glycosaminoglycans having an average molecular weight (weight average) of up to 50 kD, or mixtures thereof.

Crosslinked glycosaminoglycans can for example be covalently or non-covalently crosslinked. The preparation of the crosslinked glycosaminoglycans can take place in a known manner. In this case, the covalent crosslinking generally takes place by crosslinking with bifunctional reactive agents, such as diepoxyoctane, BDDE, divinyl sulfone, glutaraldehyde or carbodiimide, via bifunctional amino acids, e.g. lysine, protamine or albumin. It is also possible, however, for crosslinking to be established for example via an amide, ester or ether bond. Further suitable reagents for covalent crosslinking of glycosaminoglycans are ethylene glycol or 1-4-butandiol-diglycidyl ether, divinyl sulfone, photo-crosslinking reagents such as ethyl eosin, hydrazides such as bishydrazide, trishydrazide, and polyvalent hydrazide compounds. Furthermore, intra- and/or intermolecularly esterified glycosaminoglycans, or glycosaminoglycans crosslinked with hexamethylene diamine, can also be used. Autocatalytic processes or non-covalent crosslinking using multivalent metal ions, such as iron, copper, zinc, calcium, magnesium, manganese, barium and other chelating metal ions are particularly preferred.

In application, the molecular weight is important, and, in the case of crosslinked glycosaminoglycans, the cross-linking level, which is for example in the range of from 0.1% to 10%, on a molar basis, without being limited thereto. In general, it can be noted that, in the case of long-chain glycosaminoglycans, a lower cross-linking level is sufficient in order to obtain a gelatinous matrix, while in the case of short-chain glycosaminoglycans a higher cross-linking level is required in order to obtain comparable properties.

The administration of the hydroxymethyl donor, e.g. the hydroxymethyl group-containing glycosaminoglycan, can in principle take place in any desired manner, provided that this is suitable for treating the disease in question. Systematic or local administration is conceivable. In many cases, local administration takes place, in the region of the affected body site.

A hydroxymethyl group donor, e.g. a containing glycosaminoglycan, can be provided, for use in the treatment of virus infections, in the form of a pharmaceutical composition, in which one or more hydroxymethyl group donors, e.g. hydroxymethyl group-containing glycosaminoglycans, are contained in an amount of preferably 0.01 to 20 wt. %, based on the total pharmaceutical composition, in particular in an amount of 0.01 to 5 wt. %, and particularly preferably in an amount of from 0.01 to 1 wt. %.

The pharmaceutical compositions according to the invention can contain, as pharmaceutical excipients, e.g. means for pH adjustment, stabilising agents, antioxidants, solubilisers, penetration enhancing agents, preservatives and/or gelling agents, as are typically used in such compositions. They are used in the amounts conventional in such preparations.

The combination of the active agent according to the invention with other active agents (e.g. corticoids, sympathomimetic drugs, parasympatholytics, and/or leukotriene receptor antagonists) is a particular embodiment and targets the co-treatment of concomitant respiratory tract diseases of non-viral origin, such as bronchial asthma. A combination with Na-citrate is possible for co-treatment of a coagulopathy.

Additives are also of importance, such as divalent or trivalent metal ions, which can have a crosslinking and stabilising effect by means of chelating, and which can also accelerate the degradation of the active ingredients.

In tissue, the degradtion of glycosaminoglycans takes place naturally, by means of a plurality of different enzymes or by means of oxygen radicals. Hyaluronic acid is degraded by hyaluronidases or oxygen radicals. Therefore, additives are also of importance which have an inhibiting effect on enzymes such as hyaluronidase (heparin, indomethacin and/or salicylate) and those which prevent the oxidative degradation in tissue, as what are known as radical quenchers (vitamins A, E and/or C).

In a particularly preferred embodiment of the invention, for treating of preventing virus infections, a mixture of long-chain glycosaminoglycans (≥200 kD) with short-chain glycosaminoglycans (e.g. dimers, trimers, tetramers, pentamers or hexamers of the repetitive disaccharide units or of up to 50 kD), or also mixtures of the above-mentioned with crosslinked glycosaminoglycans is used. A further particularly preferred embodiment is mixtures of crosslinked and non-crosslinked glycosaminoglycans.

The preparations according to the invention are preferably applied to the surfaces of the respiratory tract, e.g. nose, mouth, throat, windpipe, bronchial tubes and/or lungs. If mucous membranes of the respiratory tract are affected by an infectious disease, in particular treatment can take place using an aerosol as an inhalation solution. In this case, the aerosols can be present in different droplet sizes of e.g. 0.5 micrometres to 100 micrometres, preferably from 5 to 30 micrometres. Methods for producing suitable aerosols for application for the respiratory tract are known. The propellant gas may be oxygen for treating concomitant breathlessness. Physiological saline solutions (NaCl, Ringer's solution, acetate solutions), polyalcohols (gylcerin), lipid emulsions are possible as support media for the active agent according to the invention.

Other application preparations in the form of a spray, such as nasal sprays, gels, which can be introduced into the naso-oropharyngeal region, possibly also chewing gums, lozenges, chewable tablets, which release the active agent into the oral cavity, rinsing or drip solutions, e.g. eye drops, are also conceivable. Intravenous, systematic application is also possible.

Administering the preparations according to the invention makes it possible to prevent or slow the establishment of viruses, e.g. coronaviruses, in the respiratory tract, e.g. in the upper respiratory tract including the mouth and throat. The spread of the virus in the lower respiratory tract or the lungs can also be prevented or slowed in the case of pre-existing infections. In particular, inactivation of the viruses in the respiratory tract of an infected person can prevent the transmission of contagious pathogens to healthy individuals. Furthermore, virions which have already penetrated into the organism and have been inactivated by contact with the preparation according to the invention—within the meaning of a vaccination with attenuated pathogens—can also trigger an immune response, which leads to the production of specific antibodies.

The present invention preferably relates to the administration of the composition to human patients, in whom there is already a viral infection, e.g. a coronavirus infection. The administration is particularly preferably performed on patients in whom the infection is in an early stage, in particular in a stage in which the pharynx and optionally the upper respiratory tract, but not yet the lungs, are affected.

In the case of a coronavirus infection, first of all the upper respiratory tract is affected. The multiplication of the virus in the epithelial cells already takes place here. This stage lasts approximately 1 week. Upon testing, the result of the throat swab is positive. The infection then moves into the lower respiratory tract and causes the actually life-threatening viral pneumonia. If the multiplication of the coronavirus is already initially stemmed successfully, by means of the agent according to the invention, the exacerbation of the disease can be prevented, or the time window for developing immunity can be increased. Likewise, the infection and contamination of healthy individuals can be prevented.

It is furthermore known that coronaviruses can also affect the gastrointestinal tract of a person. Consequently, the described treatment principle or the described preparations can be intended for peroral administration using a suitable carrier. In this case, a particular preparation form is a hydroxymethyl-modified highly crosslinked hyaluronic acid gel, or a hydroxymethyl-modified polysaccharide such as starch and cellulose.

The mentioned treatment principles are furthermore also used in veterinary medicine.

The preparation of a composition according to the invention can take place in a manner that is conventional per se for the preparation of such compositions, and is generally known. In this case, the sequence of the mixing of the individual components is generally not critical.

The type, dose and frequency of the administration of the composition according to the invention, and the constitution (e.g. viscosity, cross-linking level, active agent content, etc.) are determined in particular by the type and severity of the disease, and by the age of the patient, and the site and the type of the application.

The type of the treatment and the frequency of the application are also determined in particular by the individual responses of the people to be treated. An application of solutions preferably takes place once or multiple times a day.

The invention also includes mixtures of a hydroxymethyl group-containing glycosaminoglycans with other glycosaminoglycans in crosslinked and/or non-crosslinked form. Mixtures of hydroxymethyl group-containing hyaluronic acid and heparin are preferred. Mixtures of hydroxymethyl group-containing hyaluronic acid and positively charged glycosaminoglycans such as chitosamine are furthermore preferred.

The preparation of a hydroxymethyl group-containing glycosaminoglycan is performed as described in WO 2012/168462 and preferably comprises the steps of:

(i) providing glycosaminoglycan and
(ii) substituting one or more amino groups of the glycosaminoglycan with hydroxymethyl groups.

Preferably hyaluronic acid is used as the glycosaminoglycan starting product in step (i).

In step (ii), the glycosaminoglycan is then substituted with hydroxymethyl groups, by chemical treatment. Step (ii) can be performed together with step (i) or thereafter. Step (ii) can for example comprise the reaction of the glycosaminoglycan with formaldehyde or an agent that releases under the reaction conditions formaldehyde, such as taurolidine,

Subsequently, the glycosaminoglycan modified with hydroxymethyl groups can be purified in a further step (iii). Excess formaldehyde or residues of formaldehyde-releasing reagents from step (ii) are removed in the process. The purification can be performed for example by precipitation with e.g. alcohols or salts, by chromatography methods, dialysis methods, vacuum extraction, and/or freeze-drying.

Optionally a further step (iv) is subsequently carried out, in which the glycosaminoglycan, substituted with hydroxymethyl groups, is crosslinked.

In principle, the crosslinking can also be performed first, followed by the introduction of hydroxymethyl groups. The crosslinking can, as described above, take place according to methods known in the prior art.

In a further preferred embodiment of the invention, a hydroxymethyl group-containing glycosaminoglycan can subsequently be combined with one or more further active agents and/or additives. Examples for such further active agents and additives are explained above.

A further aspect of the invention relates to a combination of hydroxymethyl group-containing glycosaminoglycan with taurolidine, e.g. with a taurolidine solution, e.g. a 0.01 to 1.0% (w/v), in particular a 0.01 to 0.5% (w/v) or a 0.1 to 0.2% (w/v) taurolidine solution. In this case, preferably hyaluronic acid is used as the glycosaminoglycan, it being possible for the molecular weight of the hyaluronic acid to be for example between 100,000 and 10,000,000 Daltons.

The active agent according to the invention should be stored in a closed container, since hydroxymethyl residues can escape, as formaldehyde, by means of an equilibrium reaction in aqueous solution, and thus the effectiveness is lost.

A preparation of crosslinked hyaluronic acid, which is isolated from cockscomb by means of formaldehyde (trade name Lubravisc; company: Bohus, Sweden), is authorised in the EU for treatment of degenerative joint diseases in humans and animals. Although the preparation with formaldehyde necessarily leads to a modification of the hyaluronic acid, and the agent thus has an anti-inflammatory, and therefore medicinal, effect by way of blocking serine proteases in the joint, it is registered as a medical product.

The isolation of hyaluronic acid from streptococci takes place without a formaldehyde step. The end product is free of hydroxymethyl groups, with the result that an additional hydroxymethylation step has to take place in order to achieve an antiviral effect.

Should the regulatory authorities come to an analogous decision for the present invention, an accelerated approval process could be expected. This would be desirable in order to contain the impending coronavirus pandemic.

A further aspect of the invention relates to a pharmaceutical preparation for oral or nasal administration, in particular for administration as an aerosol or nasal spray, which contains a hydroxymethyl group donor as an active agent, optionally in a pharmaceutically acceptable carrier, expediently in a powdery carrier or in a liquid, e.g. an aqueous, carrier, such as a physiological saline solution. This preparation is intended in particular for application in the respiratory tract, and can in particular be used for one of the above-described medical indicators, including an infection with SARS-CoV-2, or for preventing an infection with SARS-CoV-2.

In a preferred embodiment, the pharmaceutical preparation contains taurolidine as the active agent, in particular in a concentration of from 0.01 to 1.0% (w/v), in particular from 0.01 to 0.5% (w/v), or from 0.1 to 0.2% (w/v), in a suitable, e.g. aqueous, carrier.

In a further preferred embodiment, the pharmaceutical preparation contains hydroxymethyl-modified hyaluronic acid, in particular in a concentration of from 0.01 to 1.0% (w/v), in particular from 0.01 to 0.5% (w/v), or from 0.1 to 0.2% (w/v), in a suitable carrier, e.g. an aqueous carrier.

A further aspect of the invention relates to a pharmaceutical preparation for peroral administration, in particular for administration as a gel, tablet or capsule, which contains a hydroxymethyl group donor as an active agent, optionally in a pharmaceutically acceptable carrier, expediently in a solid carrier or in a liquid carrier, e.g. an aqueous carrier, such as a physiological saline solution. This preparation is intended in particular for application in the digestive tract, and can in particular be used for one of the above-described medical indicators, including an infection with SARS-CoV-2, or for preventing an infection with SARS-CoV-2. Optionally, this preparation is provided with a casing, e.g. a gastric acid-resistant coating, such that the active agent can be released in the stomach, e.g. in the duodenum, jejunum and/or colon.

In a preferred embodiment, the pharmaceutical preparation contains a hydroxymethyl-modified hyaluronic acid, e.g. a hydroxymethyl-modified crosslinked hyaluronic acid gel or a modified polysaccharide such as starch and cellulose in a suitable carrier, e.g. a solid or a liquid carrier.

In order to improve the tolerability and to reduce undesired effects, as well as to control the pharmacological effect of the active substance, the aqueous carriers can, by means of physiological electrolytes and/or buffer systems, accordingly have a physiological osmolarity and buffered pH that is preferably in the physiological range, e.g. between 6.0 and 8.0.

A further subject of the invention relates to the use of a hydroxymethyl group donor or a pharmaceutical preparation as described above, for reducing and/or preventing side-effects of a vaccination against a viral infection, in particular an infection with coronaviruses such as SARS-CoV-2. The vaccination is preferably a vaccination with a genetic vaccine, i.e. a vaccine which contains a nucleic acid, coding for a viral antigen, as the active agent, particularly preferably with a genetic vaccine against a coronavirus such as SARS-CoV-2. The genetic vaccine is particularly preferably an mRNA or a vector active agent, in particular a vaccine which contains the genetic information for a coronavirus spike protein.

For this purpose, the hydroxymethyl group donor or a pharmaceutical preparation which contains the hydroxymethyl group donor as the active agent is administered to a person who is to be vaccinated or is vaccinated. The administration can take place in a prophylactic manner together with the vaccination, preferably at a time spacing of approximately 24 h or less, in particular of approximately 12 h or less, from the vaccination, or, alternatively, therapeutically after the appearance of symptoms. In this case, the administration can take place according to all the above-mentioned methods, inhalative and intraperitoneal, e.g. intravenous, administration being preferred. Taurolidine is preferably used as the as hydroxymethyl group donor.

A further subject of the invention relates to an in vitro method for inactivating viruses, in particular enveloped viruses such as coronaviruses, e.g. SARS-CoV-2 viruses, paramyxoviruses, e.g. RS viruses, or orthomyxoviruses, e.g. influenza viruses, comprising the treatment of a preparation of enveloped viruses with a hydroxymethyl group donor under conditions leading to the killing and/or attenuation of the viruses in the treated preparation.

The treatment preferably achieves a reduction in the cytopathic effect (CPE) of the viruses in the treated preparation, by a factor of 10, 10², 10³, 10⁴, 10⁵, 10⁶ or more. Suitable standard tests are available to a person skilled in the art for determining the CPE of viruses. The CPE of SARS-CoV-2 can for example be determined by a test, as described by K. Gorshkov et al. “The SARS-CoV-2 Cytopathic Effect is Blocked by Lysosome Alkalizing Small Molecules”, ACS Infect Dis. (December 2020): acsinfectdis.0c00349. Optionally, the inactivation step according to the invention can be combined with further inactivation steps such as heating, acid treatment and/or irradiation, e.g. with UV or ionising rays.

The inactivation step is typically carried out by treating the virus preparations in an aqueous medium, to which the hydroxymethyl group donor is added. The medium can optionally also contain organic solvents, miscible with water, such as DMSO. The final concentration of the hydroxymethyl group donor in the preparation can for example be selected as described above. Optionally, however, the hydroxymethyl group donor can also be used in a higher concentration. When using taurolidine, for example concentrations of 0.01-10% (w/v) or 0.1-3% (w/v) are suitable. Expediently, the treatment is carried out at a temperature in the range of from approximately 5° C. to approximately 50° C. The duration of the treatment is typically at least 5 min, preferably at least 10 min.

The virus preparation treated with a hydroxymethyl group donor can optionally be used as a vaccine, which can be administered in a suitable manner, e.g. by injection and/or by inhalation in the form of an aerosol.

Yet a further subject of the invention is an inactivated preparation of viruses, in particular enveloped viruses such as coronaviruses, e.g. SARS-CoV-2 viruses, paramyxoviruses, e.g. RS viruses, or orthomyxoviruses, e.g. influenza viruses, which has been modified with a hydroxymethyl group donor.

The cytopathic activity of the treated virus preparations is expediently reduced by at least a factor of 10, 10², 10³, 10⁴, 10⁵, or 10⁶, compared with an untreated virus preparation.

The inactivated virus preparation according to the invention can be used for example as a vaccine for human and/or animal medicine.

### PILOT STUDY

The clinical effectiveness and/or the antiviral effect of taurolidine and hydroxymethyl-modified hyaluronic acid were checked on the basis of a pilot study with voluntary participants, in the sense of an individual curative treatment.

Overall, the study was carried out on 19 patients with symptoms of a respiratory tract disease or colds and flu.

Of these, 6 tested positive and 4 negative for SARS-CoV-2 (point of care test, POCT). In the case of the remaining participants, the clinical symptoms were the indication for the start of treatment.

The taurolidine treatment was carried out by inhaling 1-2 ml aerosol comprising 0.5% taurolidine, by means of an ultrasonic nebuliser or a compressed air nebuliser. (According to the manufacturer's specifications, these devices generate aerosols having a predominant particle size of 5 micrometres). Depending on the symptoms, the application took place between twice and 4 times a day. Taurolidine was extracted from a commercial 2% stock solution (TauroNova by Tauropharm), and diluted with physiological saline solution or with a buffered saline infusion solution (pH 7.2).

Alternatively, a 0.5% solution with hydroxymethyl-modified hyaluronic acid was used as an aerosol for inhalation or as a nasal spray. The approved commercial product Lubravisc, having a hyaluronic acid content of 2%, was diluted with physiological saline solution to 0.5%. The hyaluronic acid modification was achieved by isolation from cockscomb, using formaldehyde. In this case, a hydroxymethyl group transfer to the hyaluronic acid takes place.

In each case the time interval from the start of treatment to the easing of the symptoms was measured. None of the participants were hospitalised.

### EXAMPLE 1

A 64-year-old male participant had a positive SARS-CoV-2 antigen test. 10 days later, he became unwell with a fever of over 38° C., muscle pain, and weakness. He then inhaled 0.5% taurolidine twice a day. 4 days later, the body temperature and wellbeing became normal.

### EXAMPLE 2

A 32-year-old participant had close domestic contact with a person verifiably suffering from Covid-19. When, shortly after, disease symptoms such as increasing body temperature, weakness and back muscle pain appeared, the POCT was positive. Accordingly, he began to inhale taurolidine 0.5% three times a day. The symptoms reduced within 48 hours, until restoration to health was achieved.

### EXAMPLE 3

A 52-year-old participant became unwell with a cough, stinging eyes, fever and lung pain. She had a positive POCT test for Covid-19. Thereupon, she inhaled taurolidine 0.5% 3 times a day. On day 4, following the start of treatment, she was symptom-free.

### EXAMPLE 4

Two participants, a couple, male and female, aged 72 and 69, respectively, became infected from a 96-year-old close relative who had tested positive for SARS-CoV-2, and were quarantined after also having had a positive test result. Over this time, they both developed dry coughs and began to inhale taurolidine 0.5% three times a day. The coughing stopped, in both participants, after 3 days. No further disease symptoms appeared.

### EXAMPLE 5

A 13-year-old participant was living in close domestic contact with his mother, who was unwell and tested positive for SARS-Cov-2. He developed a sore throat, and subsequently also tested positive by POCT. Thereafter, he inhaled taurolidine 0.5% twice a day. On the 4th day he was symptom-free.

### EXAMPLE 6

2 female and 2 male participants, aged from 27 to 64, members of one family, who had had close contact with two relatives who had tested positive for Covid-19 and had become unwell, prophylactically inhaled taurolidine 0.5%, 3 times a day or twice a day, for 7 days, once they knew of the illness of their relatives, simultaneously with the infected relatives. The 4 participants remained symptom-free, and the POCT for SARS-CoV-2 remained negative in all cases.

### EXAMPLE 7

A 29-year old participant developed a sore throat with unspecific malaise. He inhaled modified hyaluronic acid twice a day. From the 3rd day he was symptom-free. A test for SARS-CoV-2 was not performed.

### EXAMPLE 8

A participant developed a runny nose and a sore throat. She used hyaluronic acid, as a nasal spray, twice a day. The symptoms eased on the 3rd day following the start of treatment.

### EXAMPLE 9

2 participants with cold symptoms, such as a runny nose, a cough, a sore throat inhaled taurolidine 0.5% three times a day, immediately following the appearance of the symptoms. They were symptom-free after 3 to 5 days following the start of treatment. A SARS-CoV-2 test was not carried out.

### EXAMPLE 10

A 28-year-old participant suffered from cold symptoms, such as a cough, a sore throat, and subfebrile body temperatures for 4 days. She inhaled taurolidine 0.5% once, in the evening of the 4th day, and was symptom-free the following day. A test for SARS-CoV-2 was not carried out.

1. Hydroxymethyl group donor for use in the treatment or prevention of an infection with coronaviruses, paramyxoviruses or orthomyxoviruses.

2. Hydroxymethyl group donor according to claim 1, characterised in that it is selected from hydroxymethyl group-containing glycosaminoglycans, wherein one or more amino groups and/or hydroxyl groups are substituted with hydroxymethyl, or is selected from taurolidine.

3. Hydroxymethyl group donor according to claim 2, characterised in that the glycosaminoglycan is selected from hyaluronic acid, heparin, chondroitin sulfate, chitosamine and poly-N-acetyl glucosamine, preferably hyaluronic acid, wherein in each case one or more amino groups are substituted with hydroxymethyl.

4. Hydroxymethyl group donor according to claim 2 or 3, characterised in that the glycosaminoglycan is selected from hyaluronic acid.

5. Hydroxymethyl group donor according to any of the claims from 2 to 4, characterised in that the degree of hydroxymethylation is in the range of 200:1 (0.5%) to 1:1 (100%), preferably 100:1 (1%) to 10:1 (10%).

6. Hydroxymethyl group donor according to any of the claims from 2 to 5, characterised in that the hydroxymethyl group-containing glycosaminoglycan is present in non-crosslinked form, the glycosaminoglycan in particular being selected from
(i) long-chain glycosaminoglycans with an average molecular weight (weight average) of at least 200 kD and
(ii) short-chain glycosaminoglycans with an average molecular weight (weight average) of up to 50 kD
or mixtures thereof.

7. Hydroxymethyl group donor according to any of the claims from 2 to 5, characterised in that the hydroxymethyl group-containing glycosaminoglycan is present in crosslinked form, the cross-linking level in particular being in the range of 0.1% to 10%.

8. Hydroxymethyl group donor according to any of the claims from 2 to 7, characterised in that the hydroxymethyl group-containing glycosaminoglycan is present as a mixture of non-crosslinked and crosslinked glycosaminoglycans.

9. Hydroxymethyl group donor according to claim 1 or 2, characterised in that it is taurolidine.

10. Hydroxymethyl group donor according to any of the preceding claims, characterised in that it is present in combination with at least one inhibitor of glycosaminoglycan degradation, the inhibitor of glycosaminoglycan degradation being selected in particular from heparin, indomethacin, salicylates, radical quenchers, such as vitamin A, C or E, and mixtures thereof.

11. Hydroxymethyl group donor according to any of the preceding claims for use in human medicine or veterinary medicine.

12. Hydroxymethyl group donor according to any of the preceding claims for local administration.

13. Hydroxymethyl group donor according to any of the preceding claims for administration on surfaces of the upper respiratory tract.

14. Hydroxymethyl group donor according to any of the preceding claims, characterised in that it is present as an inhalable preparation, in particular as an aerosol, or as a nasal spray.

15. Hydroxymethyl group donor according to any of the preceding claims for administration to patients with an infection of the upper and/or lower respiratory tract.

16. Hydroxymethyl group donor according to any of the preceding claims for treating an infection by coronaviruses, in particular for treating patients with an infection by SARS-CoV-2.

17. Method for treating or preventing an infection with coronaviruses, paramyxoviruses or influenza viruses, characterised in that a subject to be treated, in particular a human subject, is administered a preparation which contains a hydroxymethyl group donor as defined in any of claims 1 to 10, in an amount sufficient for treating the disease.

18. Hydroxymethyl group donor for use as an active agent for treating and preventing an inflammatory disease of the respiratory tract, for example COPD, ARDS or cystic fibrosis, in particular an inflammatory disease of the respiratory tract associated with a viral infection.

19. Method for treating or preventing an inflammatory disease of the respiratory tract, for example COPD, ARDS or cystic fibrosis, in particular an inflammatory disease of the respiratory tract associated with a viral infection, characterised in that a subject to be treated, in particular a human subject, is administered a preparation which contains a hydroxymethyl group donor as defined in any of claims 1 to 10, in an amount sufficient for treating the disease.

20. Pharmaceutical preparation for oral or nasal administration, in particular for administration as an aerosol or nasal spray, characterised in that it contains a hydroxymethyl group donor as an active agent in a pharmaceutically acceptable carrier.

21. Pharmaceutical preparation according to claim 20, characterised in that it contains taurolidine, in particular in a concentration of 0.01 to 1.0% (w/v), in particular 0.01 to 0.5% (w/v), or 0.1 to 0.2% (w/v), in an aqueous carrier.

22. Pharmaceutical preparation according to claim 20, characterised in that it contains hydroxymethyl-modified hyaluronic acid, in particular in a concentration of 0.01 to 1.0% (w/v), in particular 0.01 to 0.5% (w/v), or 0.1 to 0.2% (w/v), in an aqueous carrier.

23. Pharmaceutical preparation according to any of the claims from 20 to 22 for application in the respiratory tract.

24. Pharmaceutical preparation for peroral administration, in particular for administration as a gel, tablet or capsule, characterised in that it contains a hydroxymethyl group donor as an active agent in a pharmaceutically acceptable carrier.

25. Pharmaceutical preparation according to claim 24, characterised in that it is provided with a coating, e.g. a gastric acid-resistant coating.

26. Pharmaceutical preparation according to claim 24 or 25, characterised in that it contains a hydroxymethyl-modified hyaluronic acid, e.g. a hydroxymethyl-modified crosslinked hyaluronic acid gel or a modified polysaccharide such as starch and cellulose in a solid or a liquid carrier.

27. Pharmaceutical preparation according to any of the claims from 24 to 26 for application in the digestive tract.

28. Hydroxymethyl group donor or pharmaceutical preparation, which contains a hydroxymethyl group donor as the active agent, in a method for reducing and/or preventing side-effects of a vaccination against a viral infection.

29. Hydroxymethyl group donor or pharmaceutical preparation, which contains a hydroxymethyl group donor as the active agent, for use according to claim 28, in case of a vaccination against coronaviruses, in particular with SARS-CoV-2.

30. Hydroxymethyl group donor or pharmaceutical preparation, which contains a hydroxymethyl group donor as the active agent, for use according to claim 28 or 29, in case of a vaccination with a genetic vaccine, in particular with a genetic vaccine against a coronavirus such as SARS-CoV-2.

31. Hydroxymethyl group donor or pharmaceutical preparation, which contains a hydroxymethyl group donor as the active agent, for use according to any of the claims from 28-30, in the case of a vaccination with an mRNA or a vector active agent.

32. Hydroxymethyl group donor or pharmaceutical preparation, which contains a hydroxymethyl group donor as the active agent, for use according to any of the claims from 28-31, in the case of a vaccination with a vaccine that contains the genetic information for a coronavirus spike protein.

33. Hydroxymethyl group donor or pharmaceutical preparation, which contains a hydroxymethyl group donor as the active agent, for use according to any of the claims from 28-32 for intraperitoneal, e.g. intravenous, or inhalative administration.

34. In vitro method for inactivating viruses, in particular enveloped viruses such as coronaviruses, e.g. SARS-CoV-2 viruses, paramyxoviruses, e.g. RS viruses, or orthomyxoviruses, e.g. influenza viruses, comprising the treatment of a preparation of viruses, in particular enveloped viruses, with a hydroxymethyl group donor under conditions leading to the killing and/or attenuation of the viruses in the treated preparation.

35. Virus preparation, in particular of enveloped viruses such as coronaviruses, e.g. SARS-CoV-2 viruses, paramyxoviruses, e.g. RS viruses, or orthomyxoviruses, e.g. influenza viruses, characterised in that it contains viruses killed and/or attenuated by a hydroxymethyl group donor.

36. Use of a virus preparation, prepared according to claim 34, or a virus preparation according to claim 35, as a vaccine.
